(19) United States
(12) Patent Application Publication
(10) Pub. No.: US 2025/0270608 A1
(43) Pub. Date: Aug. 28, 2025

(54) SYNTHESIS AND STORAGE OF DNA HAVING ATTACHED NANOSTRUCTURE

(71) Applicant: Seagate Technology LLC, Fremont, CA (US)

(72) Inventors: Mengdi BAO, Shakopee, MN (US); Gemma Roselle MENDONSA, Minneapolis, MN (US); Riyan Alex MENDONSA, Minneapolis, MN (US)

(21) Appl. No.: 19/067,514

(22) Filed: Feb. 28, 2025

Related U.S. Application Data
(60) Provisional application No. 63/559,105, filed on Feb. 28, 2024.

(51) Int. Cl. C12P 19/34; C12N 15/11; C12N 15/113

(52) U.S. Cl. CPC C12P 19/34; C12N 15/111; C12N 15/113; C12N 2310/127; C12N 2310/20

(57) ABSTRACT

Using a nanostructure as a support structure for DNA, particular for binary data-encoding DNA, both during the writing or assembly step and during long-term storage. The nanostructure may be a nanoparticle, such as a magnetic nanoparticle (MNP), e.g., a silica-based MNP. The nanostructure serves as a carrier for DNA storage due to the high-aspect-ratio surface area of the nanostructure, which enables high-density DNA immobilization on the surfaces. When silica nanostructures are used, DNA encapsulated within the silica endures exposure to high temperatures, oxygen radicals, and ultraviolet light without significant damage, thus, supporting long term storage of the DNA. Magnetic nanostructures facilitate, via magnetic separation, buffer exchanges and removal of unligated strands.

### CROSS-REFERENCE

This application claims priority to U.S. provisional application 63/559,105 filed Feb. 28, 2024, the entire discourse of which is incorporated herein by reference for all purposes.

### BACKGROUND

DNA data storage has demonstrated remarkable advantages in terms of digital information storage, surpassing traditional mediums like magnetic tapes in storage density, retention duration, and energy efficiency. However, it must be acknowledged that DNA is susceptible to external environmental factors when not adequately protected by storage systems. These factors include exposure to reactive oxygen, ionizing radiation, heat, chemicals, and nuclease attacks.

Additionally, many current DNA writing approaches necessitates a column-based purification step to exchange buffers and enable future writing. However, column purification is a labor-intensive and costly process.

There is a pressing need to develop a storage system for data encoded DNA that supports buffer exchange during the DNA writing step while also protecting DNA from external environmental factors during storage.

### SUMMARY

This disclosure is directed to using a nanostructure as a support structure for data-encoded DNA, both during the writing or assembly step and during long-term storage. The selection of the nanostructure, such as nanoparticles, is driven by their substantial surface area and small size, enabling the efficient immobilization of DNA on their surfaces. A suitable nanostructure material is silica, due to its chemical and thermal stability, and its outstanding barrier properties.

One particular implementation described herein is using a nanostructure as a support structure for DNA, particular for binary data-encoding DNA, both during the writing or assembly step and during long-term storage. The nanostructure may be a nanoparticle, such as a magnetic nanoparticle (MNP) having a silica surface.

Another particular implementation described herein is a method of assembling a DNA strand encoding data, the method including immobilizing a first DNA oligo to a nanostructure to form a first labeled strand, ligating a second DNA oligo to that first labeled strand to form a second labeled strand, and releasing the nanostructure from the second labeled strand to form a second strand.

Another particular implementation described herein is a method of assembling a DNA strand encoding data, the method including ligating a first immobilized DNA oligo to a second DNA oligo via a ligation process to form a labeled strand, cleaving a nanostructure from the labeled strand to form a first strand, and ligating a second immobilized DNA oligo to the first strand via another ligation process to form a second labeled strand.

These implementations can utilize DNAzyme ligation for the ligating steps.

Additionally or alternately, the nanostructure can be a magnetic nanoparticle.

Another particular implementation described herein is, after assembling a data-encoding DNA strand, providing a silica nanoparticle on an end of the DNA strand and storing the DNA strand until reading (sequencing) the data.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. These and various other features and advantages will be apparent from a reading of the following detailed description.

### BRIEF DESCRIPTION OF THE DRAWING

The described technology is best understood from the following Detailed Description describing various implementations read in connection with the accompanying drawing.

FIG. 1A shows, stepwise, a DNAzyme ligation method using a nanoparticle (NP).

FIG. 1B is a flowchart showing, stepwise, the DNAzyme ligation method of FIG. 1A.

### DETAILED DESCRIPTION

DNA is an emerging technology for data storage. When using DNA as a binary data storage gene, a bit pattern (e.g., 00, 01, 10, 11) is assigned to each nucleotide (A, C, G, T), thus providing a gene encoding the desired data. In one example, A=00, C=10, G=01, and T=11. The DNA strand is assembled, or built, so that the nucleotides are in the desired order to encode the desired data. Pre-prepared oligos may be used to assemble the DNA strand, the pre-prepared oligos already having the nucleotides arranged in the desired pattern for the desired data. The resulting DNA strand, encoding the binary data in its order of nucleotides, is then stored until the data is to be retrieved, or read. Numerous methods for assembling the data- encoding DNA strand have been described, as well as numerous methods for reading the data, some of which disassemble the strand into individual nucleotides. Better methods are needed. Better solutions are also needed for storing the strands after being assembled but before being read.

As indicated above, this disclosure provides a method that utilizes nanostructured materials, such as silica nanoparticles having a magnetic element (e.g., a magnetic nanoparticle, or MNP), as platforms for both the assembly of data-storage DNA during the writing step and for long-term storage. The nanostructures, such as nanoparticles, have substantial surface area and a small size, enabling the efficient immobilization of DNA on the surfaces. Multiple DNA strands can be immobilized on a nanostructure.

In general, any nanostructure material can be used, however magnetic nanostructures facilitate buffer changes and exchanges and removal of unligated strands via magnetic separation. Examples of suitable nanostructure and/or nanoparticle materials include silica, polymers, gold, silver, titania, iron oxide, and zinc. Polymeric nanostructures are typically loaded or coated with an inert or activated material, such as carboxyl (e.g., the nanostructure having a carboxylic acid surface). The nanostructure and/or nanoparticle may be magnetic due to the base material (e.g., iron oxide) or due to an additive or coating. Silica is a preferred nanostructure and/or nanoparticle material due to its inherent chemical and thermal stability, as well as outstanding barrier properties and enduring molecular stability. A magnetic silica nanoparticle can have a magnetic base (e.g., iron oxide) with a silica coating thereon.

The nanostructure and/or nanoparticle can have any suitable shape, including spherical, rods, nanotubes, cubes, stars, or other shape. The nanostructures and/or nanoparticles have largest dimension (e.g., a diameter or length) between 10-100 nm, and in some embodiments larger than 100 nm. The larger the nanostructure/nanoparticle is, the more DNA strands can be bound to the surface of the nanostructure. In most embodiments, an end of the DNA strand is bound to the nanostructure and/or nanoparticle.

Although many different methodologies are available to construct the DNA strand (i.e., placing the nucleotides in the desired order to represent the binary data sequence), a DNAzyme ligation method is particularly suitable for building strands on a nanostructure such as a nanoparticle. In the DNAzyme ligation method, as well as other methods, pre-prepared oligos having the desired nucleotide sequence are used to build the data-encoding DNA strand.

Other chemical ligation methods and enzymatic ligation and assembly methods are also suitable methods for constructing the DNA strand on a nanostructure.

In a conventional DNAzyme DNA-ligation approach, a two-step process is used to form a DNAzyme having an S1 end and an S2 end. The initial step involves adding specific reagents to the S1 strand to activate the end phosphate group of the DNAzyme and attach the S1 strand to the DNAzyme. Following this activation, the added reagents are then removed, e.g., by column purification. In the subsequent step, the activated S1 strand is incubated with the S2 strand attached to the other end of the DNAzyme, the DNAzyme catalytic strand, and necessary reagents. This process facilitates the efficient ligation of the DNAzyme components.

Conversely, in the approach described herein, with a nanostructure or nanoparticle, separation of the molecules improves, allowing for isolation of the DNA strand from the buffer and thus facilitating buffer exchange. With a magnetic nanostructure or nanoparticle, magnetic manipulation (e.g., separation) is readily feasible.

By integrating a switch control, the immobilized DNA with altered buffers can be released and transferred to other nanoparticles-DNA conjugates for subsequent writing steps.

In the following description, reference is made to the accompanying drawing that forms a part hereof and in which is shown by way of illustration at least one specific implementation. The following description provides additional specific implementations. It is to be understood that other implementations are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

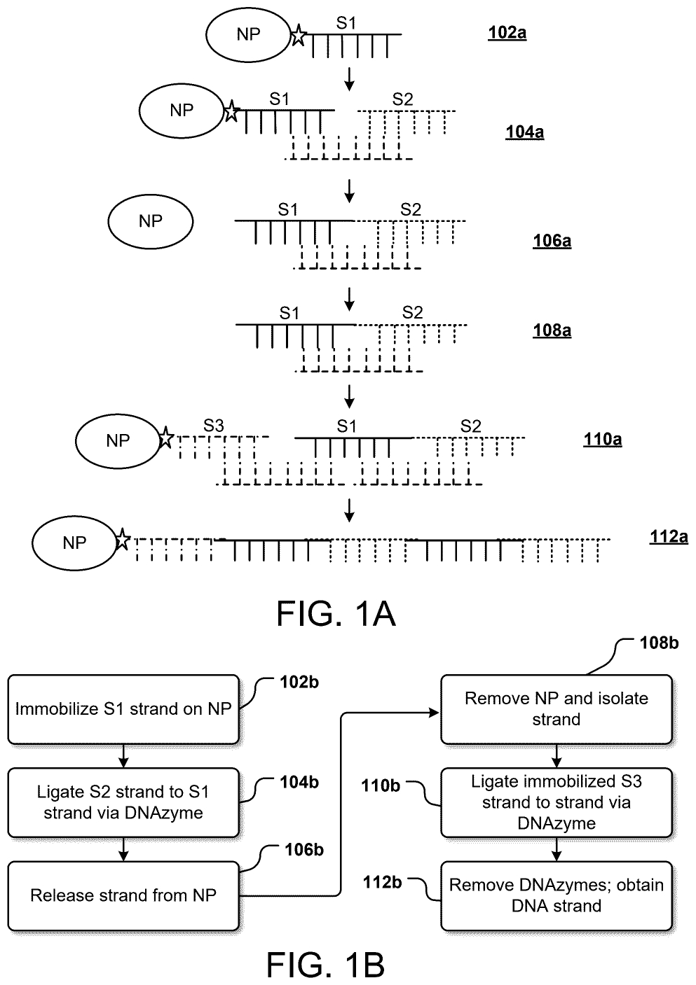

One detailed method 100 for preparing a DNA strand for encoding data, using a nanostructure such as a nanoparticle, is described below with reference to FIG. 1A and FIG. 1B, which provide a step-by-step explanation of the method 100. FIG. 1A shows, schematically, the method whereas FIG. 1B describes the steps.

The method 100 begins by immobilizing a single-stranded DNA oligo with controlled switching, labeled as S1 in step 102a of FIG. 1A, onto the surface of a nanostructure, such as a magnetic nanoparticle (NP in FIG. 1A). This immobilization of the S1 strand onto the NP (step 102b in FIG. 1B) can be achieved through various methods, such as streptavidin-biotin interactions, amino-carboxylate binding, or click chemistry.

Reagents for the initial activation step are introduced into the S1-nanoparticle conjugates. Once the activation is completed, the introduced reagents and any resulting by-products are removed through a separation step, such as a magnetic separation step.

Next, an S2 strand and catalytic strand (e.g., DNA-ligating DNAzyme) are introduced into the S1-nanoparticle conjugates, along with the necessary buffer. After incubation, e.g., at room temperature, the S1 strand is ligated to the S2 strand (step 104a, 104b).

With the switched-on control, the S1-S2 strand is released from the nanoparticles (step 106).

The S1-S2 strand is transferred to another container without the nanoparticles (step 108a, 108b); if the nanoparticle is magnetic, this may be via magnetic separation.

This S1-S2 strand is then ligated to an S3 strand that is immobilized onto a NP; this NP may be the same NP, the same type or different than the original NP. A buffer exchange is performed, and the conjugates are ligated with the S1-S2 strand (step 110a, 110b).

This iterative process can be repeated to accomplish multiple DNA writing steps. Ultimately, a long DNA molecule is formed (step 112a, 112b). The NP can be removed from the DNA strand or left immobilized thereon.

The long DNA molecule can be stored as shown in step 112a, immobilized onto a nanostructure, In some implementations, the NP can be removed and the DNA strand immobilized on a silica-based nanoparticle for stable long-term storage, e.g., if a silica-based nanoparticle was not used for the synthesis. Silica has inherent chemical and thermal stability, as well as outstanding barrier properties and enduring molecular stability. Multiple DNA strands can be immobilized on a single nanostructure, depending on the size of the nanostructure, the length of the DNA strands, and the binding chemistry used to conjugate the DNA strand and the nanostructure.

Various approaches can be used to enable switch-on control for the release of the ligated S1-S2 strands from magnetic nanoparticles, as illustrated in step 104a to step 106a, 106b. One strategy involves incorporating a photocleavable linker into the S1 strand. This photocleavable linker is sensitive to UV light, allowing for precise cleavage of the strand at a specified site upon UV light illumination. Another approach leverages CRISPR-Cas technology for sequence-specific cleavage. Specifically, the S1 strand can be designed with a complementary sequence to the CRISPR RNA (crRNA), leading to cleavage in the presence of Cas enzyme and crRNA. Additionally, DNAzyme-based cleavage serves as another tool for sequence-specific cleavage, where the DNAzyme strand can cleave the substrate strand in the presence of a specific metal ion. These versatile approaches provide controlled mechanisms for the targeted release of the S1-S2 strand from magnetic nanoparticles.

By using a nanostructure using the assembly or writing process, the nanostructures serve as carriers for DNA storage due to their high-aspect-ratio surface area, which enable high-density DNA immobilization on their surfaces. When silica nanostructures are used, DNA encapsulated within silica has proven to endure exposure to high temperatures, oxygen radicals, and ultraviolet light without significant damage, thus, including silica-based nanostructure supports long term storage of the DNA.

Magnetic nanostructures (e.g., particles) allow for magnetic separation for buffer exchanges, thus eliminating the need for columns. In comparison to column-based approaches, magnetic-purification not only offers cost-effectiveness but also shields DNA from potential damage caused by external environmental factors during both DNA writing steps. Further, using magnetic nanoparticles for buffer exchanges allows for automation of the process, as magnetic nanoparticles can be seamlessly integrated into automated equipment, such as automatic liquid handling systems or digital microfluidic devices, thereby enabling automated DNA writing steps.

Beyond facilitating buffer exchange, magnetic nanoparticles play a role in removing unligated strands. This additional function serves to prevent non-specific binding reactions, consequently enhancing the overall reaction yield. By leveraging the magnetic properties of MNPs, unligated strands can be efficiently separated and removed from the reaction mixture. This dual capability of buffer exchange and selective removal of unligated strands underscores the versatility of MNPs in optimizing reaction conditions and improving the specificity of the DNA writing process.

In addition to magnetic nanoparticles (e.g., silica MNPs) as platforms for immobilizing the DNA, other nanostructures and materials can also function as platforms that can execute multiple DNA writing steps and facilitate long-term storage. For example, MNPs and NPs having a gold surface provide stable long-term storage.

Furthermore, as indicated above, alternative DNA writing methods, such as Click chemistry-based ligation or enzymatic ligation, can be effectively used in place of the DNA-ligating DNAzyme,

The above specification and examples provide a complete description of the structure and use of exemplary implementations of the invention. The versatility of the proposed approach allows for compatibility with various DNA writing techniques, expanding its applicability and potential for innovation in nucleic acid storage and manipulation. The above description provides specific implementations. It is to be understood that other implementations are contemplated and may be made without departing from the scope or spirit of the present disclosure. It is to be understood that any features or details of one implementation may be utilized for any other implementation, unless contrary to the construction or configuration thereof. Any variations may be made. The above description, therefore, is not to be taken in a limiting sense. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

Spatially related terms, including but not limited to, “bottom,” “lower”, “top”, “upper”, “beneath”, “below”, “above”, “on top”, “on,” etc., if used herein, are utilized for ease of description to describe spatial relationships of an element(s) to another. Such spatially related terms encompass different orientations of the device in addition to the particular orientations depicted in the figures and described herein. For example, if a structure depicted in the figures is turned over or flipped over, portions previously described as below or beneath other elements would then be above or over those other elements.

Since many implementations of the invention can be made without departing from the spirit and scope of the invention, the invention resides in the claims hereinafter appended. Furthermore, structural features of the different implementations may be combined in yet another implementation without departing from the recited claims.

What is claimed is:

1. A method of assembling a DNA strand, the method comprising:
immobilizing a first DNA oligo to a nanostructure to form a first labeled strand;
ligating a second DNA oligo to the first labeled strand to form a second labeled strand; and
releasing the nanostructure from the second labeled strand to form a second strand.

2. The method of claim 1, wherein ligating a second DNA oligo to the first labeled strand is via DNAzyme ligation.

3. The method of claim 1 further comprising, after releasing the nanostructure from the second labeled strand to form the second strand:
ligating a third labeled strand to the second strand to form a fourth labeled strand, the third labeled strand comprising a third oligo immobilized to a nanostructure.

4. The method of claim 3, wherein ligating a third labeled strand to the second strand is via DNAzyme ligation.

5. The method of claim 3 further comprising, after ligating the third labeled strand to the second strand to form a fourth labeled strand:
releasing the nanostructure from the fourth labeled strand.

6. The method of claim 5, further comprising repeatedly ligating additional labeled strands and releasing additional nanostructures therefrom to form a final strand.

7. The method of claim 5, further comprising repeatedly ligating additional labeled strands and releasing additional nanostructures therefrom to form a semifinal strand, and then ligating one last labeled strand immobilized on a silica nanostructure to the semifinal strand.

8. The method of claim 1, wherein the nanostructure is a magnetic nanostructure.

9. The method of claim 1, wherein the nanostructure is a silica nanostructure.

10. A method of assembling a DNA strand, the method comprising:
ligating a first immobilized DNA oligo to a second DNA oligo via a ligation process to form a labeled strand;
cleaving a nanostructure from the labeled strand to form a first strand; and
ligating a second immobilized DNA oligo to the first strand via another ligation process to form a second labeled strand.

11. The method of claim 10 further comprising:
cleaving a nanostructured form the second labeled strand to form a second strand.

12. The method of claim 11 further comprising:
ligating a third immobilized DNA oligo to the second strand via another ligation process to form a third labeled strand.

13. The method of claim 10, wherein cleaving the nanostructure is by utilizing a photocleavable linker present in the first immobilized DNA oligo.

14. The method of claim 10, wherein cleaving the nanostructure is by utilizing a CRISPR-Cas technology for sequence-specific cleavage.

15. The method of claim 10, wherein cleaving the nanostructure is by utilizing DNAzyme-based cleavage.

16. The method of claim 10, wherein the nanostructure is a magnetic nanostructure.

17. The method of claim 10, wherein the nanostructure is a silica nanostructure.
